(19) United States
(12) Patent Application Publication
(10) Pub. No.: US 2007/0100019 A1
(43) Pub. Date: May 3, 2007

(54) Catalyst system for dental compositions

(76) Inventor: Fuming Sun, Middletown, DE (US)

Correspondence Address: DENTSPLY INTERNATIONAL INC, 570 WEST COLLEGE AVENUE, YORK, PA, 17404, US

(21) Appl. No.: 11/494,078

(22) Filed: Jul. 27, 2006

Related U.S. Application Data
(60) Provisional application No. 60/704,535, filed on Aug. 2, 2005.

(51) Int. Cl. A61K 6/08

(52) U.S. Cl. 523/116

(57) ABSTRACT

The present invention relates generally to dental compositions containing polymerizable resins and a catalyst system for accelerating polymerization and hardening of the resins. The composition is prepared from two parts—a base paste containing a polymerizable resin and thiourea compound that acts as a polymerization accelerator and a catalyst paste containing thermally stable hydroperoxide compounds. The dental compositions can be used as crown and bridge materials inlays, onlays, veneers, fillings, sealants, adhesives, cements, and other dental materials.

### CROSS-REFERENCE TO RELATED APPLICATIONS

This application claims the benefit of U.S. Provisional Patent Application 60/704,535 having a filing date of Aug. 2, 2005, the entire contents of which are hereby incorporated by reference.

### BACKGROUND OF THE INVENTION

1. Field of the Invention

The present invention relates generally to dental compositions containing polymerizable resins and a catalyst system for accelerating polymerization and hardening of the resins. The dental compositions can be used as crown and bridge materials, inlays, onlays, veneers, fillings, sealants, adhesives, cements, and other dental materials. More particularly, the composition can be used to make a temporary crown or bridge. The compositions have good shelf life, color stability, work time/set time, physical properties, wear-resistance and handling performance.

2. Brief Description of the Related Art

Dental restorations, such as crowns and bridges, are used to restore or replace lost tooth structure, teeth, or oral tissue. Temporary restorations are intended for use by patients for a relatively short time. For example, a dentist will often use a temporary crown, until a more permanent crown is ready to be placed in the mouth of a patient. Following this procedure, the patient will make several visits to the dental office. In the first visit, the dentist examines and prepares the tooth that will receive the crown. The dentist first takes an impression of the patient's entire dental anatomy using an impression tray or matrix filled with a paste-like material. After the dentist has taken this initial dental impression, the patient is anesthetized, and the tooth, which will receive the crown, is prepared. The dentist first removes any dental caries from the tooth using a high-speed dental bur. Then, the dentist performs “crown prep” work by filing and grinding the tooth to a core. Next, the dentist takes an impression of the prepared tooth. The dentist sends the resulting impression to a dental laboratory, which will make the permanent crown. During this first visit, the dentist places a temporary crown on the tooth to cover and protect the tooth. The temporary crown remains on the tooth until the permanent crown is ready to be mounted.

The temporary crown typically is made from a polymeric material such as an acrylic. In one method used to prepare a temporary crown, a polymerizable material is placed in a pre-made impression or plastic matrix, which then is inserted into the patient's mouth. The polymerizable material is molded over the tooth by pressing the impression thereon. Then, the impression containing the molded material, which may only be partially-cured at this point, is removed from the patient's mouth. The material is fully cured by a self (chemical)-curing, light-curing, or heat-curing mechanism, which may occur outside of the mouth, to form the ultimate temporary crown. Finally, the dental practitioner places the temporary crown over the prepared tooth and bonds the crown to the tooth using temporary dental cement. The temporary crown helps maintain the health of the tooth while the dental laboratory manufactures the permanent crown. The temporary dental cement holds the temporary crown in place until the dentist is ready to remove it and mount the permanent crown. At the second office visit, the dentist removes the temporary crown and checks the color and fit of the permanent crown. If the color and fit are satisfactory, the dentist affixes the permanent crown to the tooth using permanent dental cement.

Dental compositions containing polymerizable resins and filler particles often are used to prepare temporary crowns, bridges, and other restorations. Such dental compositions can be self (chemically)-curable, light-curable, or dual-curable. The dental compositions are cured and hardened by different chemical mechanisms to form a strong and durable material. In one example, a dentist uses a self-curing composition, which is prepared from two paste components. One component used to make the composition is a base paste and the other component is a catalyst paste. The base paste typically contains polymerizable monomers such as methacrylate or acrylate monomers; a free-radical polymerization accelerator such as a tertiary amine; and fillers such as silica, glasses, or alumina. Meanwhile, the catalyst paste typically includes a polymerizable monomer, a free-radical polymerization initiator such as dibenzoyl peroxide, and fillers.

To prepare the composition, the amine-containing base and peroxide-containing catalyst pastes are combined and mixed together. Typically, the respective pastes are stored separately in side-by-side auto-mix cartridges. When a dental professional is ready to prepare the composition, the pastes are extruded through a dispensing tip attached to the cartridges. The dispensing tip normally contains a static mixing element. As the pastes are mixed together, the catalyst system (amine and peroxide) react with each other and initiate polymerization and hardening of the composition. The polymerization process involves a reaction between the reducing agent (amine) and oxidizing agent (peroxide). This mechanism is commonly referred to as a redox mechanism.

In recent years, there have been attempts to use new catalyst systems in dental compositions. For example, Qian, published U.S. Patent Application No. 2004/0235981 describes a two-part, self-adhering dental composition comprising base and catalyst components. The composition comprises: (a) at least one acidic compound containing at least one acidic moiety selected from the group consisting of 1 where R is an alkyl or aryl group; (b) at least one polymerizable monomer without any acidic group where the polymerizable group is selected from the group consisting of an acrylate, a methacrylate and a vinyl group; (c) a substituted thiourea selected from the group consisting of 1-(2-pyridyl)-2-thiourea and 1-(2-tetrahydrofurfuryl)-2-thiourea; and (d) a hydroperoxide compound with at least one hydroperoxide group attached to a tertiary carbon. The two parts of the composition are mixed just prior to application and the mixed composition is applied to the tooth. Hardening of the composition occurs by self-curing or a combination of self-curing and photo-curing. The composition is described as being suitable for use as a cement, base/liner, pit/fissure sealant, primer, or adhesive.

Mitra et al., published U.S. Patent Application No. 2003/0166740 describes a dental composition comprising a hardenable resin system, a polymerizable reducing agent containing either urea or thiourea groups and their derivatives, and an oxidizing agent. The resin systems typically include an ethylenically unsaturated component such as acrylates and methacrylates and preferably also include an acid-functional component that is a polymer of alkenoic acids such as acrylic acid. Peroxides are described as being suitable oxidizing agents. The dental compositions can be used in sealants or adhesives, restoratives, and prostheses including crowns, bridges, veneers, inlays, and onlays, and is particularly suitable for use in water-based cements.

Although the foregoing dental compositions, as described in the prior art, may have some advantageous properties, they generally require the presence of an acid-functional component such as, for example, (meth)acrylated poly(acrylic acid) homopolymer and (meth)acrylated poly(acrylic acid-maleic acid) copolymer and (meth)acrylated poly(acrylic acid-itaconic acid) copolymer, to provide the composition with the desired storage, handling, and other properties. A dental composition that does not require using an acid-functional component would be less costly to prepare and provide other benefits. Such a composition should have properties that are similar to or improved over properties found in prior art compositions including good shelf life, color stability, and handling properties. The composition also should have good mechanical strength and wear-resistance so that the resulting crown, bride, or other restoration does not weaken or fracture easily. The present invention provides a dental composition having such properties. These and other objects, features, and advantages of the invention are evident from the following description and illustrated embodiments.

### SUMMARY OF THE INVENTION

The present invention provides a dental composition that can be used as crown and bridge materials, inlays, onlays, veneers, fillings, sealants, adhesives, cements, and other dental materials upon polymerization. The composition comprises a polymerizable compound, wherein said compound does not contain any acidic groups and a polymerization system for initiating polymerization of the composition, the system comprising a thiourea compound and hydroperoxide compound. The thiourea compound may be selected from the group consisting of thiourea; 2-pyridyl thiourea; phenolthiourea; 1-acetyl-2-thiourea; 1-allyl-2-thiourea; 1-allyl-3-(2hydroxyethyl)-2-thiourea; benzoyl thiourea; and mixtures thereof. The hydroperoxide compound may be selected from the group consisting of cumene hydroperoxide; t-amyl-hydroperoxide; 1,1,3,3-tetramethyl butyl hydroperoxide; t-butyl-hydroperoxide; and mixtures thereof. The compositions have good shelf life, color stability, work time/set time, and handling performance. Upon curing and hardening, the composition has high strength, wear resistance, and other physical properties.

### DETAILED DESCRIPTION OF THE PREFERRED EMBODIMENTS

Base Paste

The base paste, which is mixed with the catalyst paste to form the composition of this invention, comprises a blend of polymerizable compound, polymerization accelerator, and filler material. The polymerizable compound, which is used in the base and catalyst pastes, is capable of being hardened to form a polymer network. The polymerizable compound has sufficient strength and hydrolytic stability with low toxicity so that it can be used in the oral cavity.

One class of suitable polymerizable compounds contains materials having free radically active functional groups and includes monomers, oligomers, and polymers having one or more ethylenically unsaturated groups without any acidic groups. Such free radically polymerizable compounds include, but are not limited to, mono-, di- or poly-acrylates and methacrylates such as methyl acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, tetraethylene glycol di(meth)acrylate, 1,3-propanediol diacrylate, 1,3-propanediol dimethacrylate, trimethylolpropane tri(meth)acrylate, 1,2,4-butanetriol trimethacrylate, 1,4-cyclohexanediol diacrylate, 1,6-hexanediol di(meth)acrylate, pentaerythritol triacrylate, pentaerythritol tetraacrylate, pentaerythritol tetramethacrylate, sorbitol hexacrylate, 2,2-bis[4-(2-hydroxy-3-acryloyloxypropoxy)phenyl]propane; 2,2-bis[4-(2-hydroxy-3-methacryloyloxypropoxy)phenyl]propane (Bis-GMA); 2,2-bis[4-(acryloyloxy-ethoxy)phenyl]propane; 2,2-bis[4-(methacryloyloxy-ethoxy)phenyl]propane (or ethoxylated bisphenol A-dimethacrylate) (EBPADMA); urethane di(meth)acrylate (UDMA), diurethane dimethacrylate (DUDMA); polyurethane dimethacrylate (PUDMA); alkoxylated pentacrythritol tetraacrylatel; polycarbonate dimethacrylate (PCDMA); the bis-acrylates and bis-methacrylates of polyethylene glycols; copolymerizable mixtures of acrylated monomers; acrylated oligomers; (should we exclude acidic monomers to avoid IP conflict?) and vinyl compounds such as styrene, diallyl phthalate, divinyl succinate, divinyl adipate and divinylphthalate. The polymerizable compound can be used alone or mixtures of the polymerizable compounds can be used in the base and catalyst pastes.

Another class of polymerizable compounds that can be used in the base and catalyst paste contains materials having cationically active functional groups. This class includes, but is not limited to, cationically polymerizable epoxy resins, siloranes (compounds containing Si and O atoms in the rings), vinyl ethers, and the like.

In addition to the foregoing polymerizable compounds, the base and catalyst pastes may contain diluent, polymerizable monomers, for example, hydroxy alkyl methacrylates, ethylene glycol methacrylates, and diol methacrylates such as tri(ethylene glycol) dimethacrylate (TEGDMA) to reduce viscosity of the composition and make the composition more suitable for application.

It has been found that the composition of this invention can be prepared without using acid-functional compounds, and the composition still has good shelf-life, color stability, working/setting time, and other advantageous properties. That is, it is preferred that acid-functional components such as monomers, oligomers, and polymers of acrylic acid, aconitic acid, citraconic acid, fumaric acid, glutaconic acid, itaconic acid, maleic acid, mesaconic acid, methacrylic acid, and tiglic acid be added neither to the base nor catalyst pastes.

The base paste further includes a thiourea compound that acts as a polymerization accelerator. The thiourea compound contains thiourea groups or derivatives thereof. More particularly, in one embodiment, a substituted thiourea, 2-pyridyl thiourea (PTU) is used as an accelerator. The PTU is preferably present in the base paste in an amount in the range of about of 0.01 to about 10% based on total weight of the base paste, and more preferably in the range of about 0.1 to about 5 wt. %.

In another form of the composition, a mixture of PTU and an imidazole is used as the polymerization accelerator. Suitable imidazoles include, but are not limited to, 2-mercapto-1-methylimidazole (MMIZ). The weight ratio of PTU to MMIZ is preferably in the range of 10:1 to 1:10, and more preferably in the range of 5:1 to 1:5. Preferably, the PTU and MMIZ are each present in the base paste in an amount in the range of about 0.01 to about 10 wt. % and more preferably in the range of about 0.1 to about 5 wt. %. It has been found that using a mixture of PTU and MMIZ as the polymerization accelerator advantageously improves the working time/setting time and gel phase of the composition. This occurs when the base paste containing the PTU and MMIZ is mixed with a catalyst paste containing thermally stable hydroperoxide as described further below.

Mixtures of PTU and other ingredients including, but not limited to, thiourea (TU); phenolthiourea (PhTU); 1-acetyl-2-thiourea (ATU); 1-allyl-2-thiourea (ALTU); 1-allyl-3-(2-hydroxyethyl)-2-thiourea (ALHETU) and benzoyl thiourea (BTU) also can be used as the polymerization accelerator. Each ingredient is preferably present in the base paste in an amount in the range of about 0.01 to about 10 wt % and more preferably in the range of about 0.1 to about 5 wt. %. In addition, a mixture of PTU and a borate including, but not limited to, sodium tetraphenyl borate (STPB) can be used. Each ingredient is preferably present in the base paste in an amount in the range of about 0.01 to about 10 wt % and more preferably in the range of about 0.1 to about 5 wt. %.

In still another embodiment, a substituted thiourea including, but not limited to the above-described, PTU, ATU, BTU, PhTU, AlTU and AlHETU (preferably PTU or ATU) is used as the accelerator, and a metal ion or metal-containing species including, but not limited to, Cu(II), Fe(II), Fe(III), Co(II), Mn(II) and Ni(II) preferably copper acetylacetonate (CuAA) is used as the promoter. The accelerator is preferably present in the base paste in an amount in the range of about 0.01 to about 10 wt. % and more preferably in the range of about 0.1 to about 5 wt. %. The promoter is preferably present in an amount of about 0.0001 to about 1 wt. % based on base paste, and more preferably in the range of 0.001 to 0.1 wt. %. The combination of the substituted thiourea and metal-containing species significantly increases curing activity and improves the mechanical properties of the cured composition. This is accomplished by mixing the base paste containing the thiourea compound with a catalyst paste containing thermally stable hydroperoxide.

Conventional filler materials such as for, example, inorganic fillers, which can be naturally-occurring or synthetic, can be added. Such materials include, but are not limited to, silica, titanium dioxide, iron oxides, silicon nitrides, glasses such as calcium, lead, lithium, cerium, tin, zirconium, strontium, barium, and aluminum-based glasses, borosilicate glasses, strontium borosilicate, barium silicate, lithium silicate, lithium alumina silicate, kaolin, quartz, and talc. Preferably, the silica is in the form of silanized fumed silica. Preferred glass fillers are silanized barium boron aluminosilicate and silanized fluoride barium boron aluminosilicate. Organic particles such as polycarbonates and polyepoxides also can be used as fillers if desired.

The average particle size of the particles comprising the filler material is normally in the range of about 0.1 to about 10 microns and more preferably in the range of about 0.1 to about 5 microns. If a fumed silica filler material is used, the silica particles are preferably nanometer-sized. The silica particles preferably have an average diameter of less than 200 nm. The filler particles can be surface-treated with a silane compound or other coupling agent to improve bonding between the particles and resin matrix. Suitable silane compounds include, but are not limited to, gamma-methacryloxypropyltrimethoxysilane, gamma-mercaptopropyltriethoxysilane, gamma-aminopropyltrimethoxysilane, and combinations thereof.

The base paste also can include additives to provide the composition with specially desired properties. For example, antimicrobial agents; fluoride-releasing agents; flavorants; pigments; fluorescent agents; opalescent agents; ultra-violet stabilizers; anti-oxidants; viscosity modifiers, and the like can be added to the base paste.

Catalyst Paste

In general, the catalyst paste comprises a blend of a blend of polymerizable compound; polymerization initiator, and filler material.

The polymerizable compounds as described above, which are useful for adding to the base paste, also may be used in the catalyst paste; provided however, that the base and catalyst pastes contain at least one different polymerizable compound. Such compounds include, but are not limited to, 2,2-bis[4-(2-hydroxy-3-methacryloyloxypropoxy)phenyl]propane (Bis-GMA); 2,2-bis[4-(acryloyloxyethoxy)phenyl]propane; 2,2-bis[4-(methacryloyloxy-ethoxy)phenyl]propane (or ethoxylated bisphenol A-dimethacrylate) (EBPADMA); urethane di(meth)acrylate (UDMA); diurethane dimethacrylate (DUDMA); polyurethane dimethacrylate (PUDMA); and the like. The polymerizable compounds can be used alone in the catalyst paste or mixtures of the polymerizable compounds can be used. Diluent monomers as described above also can be added to the catalyst paste. These include, but are not limited to, hydroxy alkyl methacrylates, ethylene glycol methacrylates, and diol methacrylates. Preferably, the catalyst paste contains tri(ethylene glycol) dimethacrylate (TEGDMA). A thermally stable hydroperoxide compound, acting as a polymerization initiator, is added to the catalyst paste to make the composition self-curable. The hydroperoxides include, but are not limited to, cumene hydroperoxide (CHP); t-amyl-hydroperoxide (TAHP); 1,1,3,3-tetramethyl butyl hydroperoxide (TMBP); t-butyl-hydroperoxide (TBHP); 2,5-dimethyl-2,5-di(hydroperoxy) hexane (DMHP); diisopropylbenzene mono hydroperoxide (DPMH); and paramenthane hydroperoxide (PMHP). The polymerization initiator is preferably present in an amount of about 0.01 to about 10 wt. % based on weight of catalyst paste, and more preferably in the range of 0.1 to 5 wt. %.

In another form of the composition, a blend of at least two thermally stable hydroperoxides is added to the catalyst paste as the polymerization initiator. The curing activity and gel phase period can be improved by changing the weight ratio of the selected hydroperoxides. The preferred polymerization initiator comprises a blend of TAHP and CHP. Each hydroperoxide component is preferably present in an amount of about 0.01 to about 10 wt. % based on weight of catalyst paste, and more preferably in the range of 0.1 to 5 wt. %.

The catalyst paste may contain fillers and additives selected from the same group of fillers and additives used in the base paste as described above. The catalyst and base pastes may include the same fillers. For example, the base paste may include a mixture of silanated barium boron aluminosilicate glass filler and fumed silica. Alternatively, the catalyst paste may contain different fillers. Additives such as, for example, antimicrobial agents; fluoride-releasing agents; flavorants; pigments; fluorescent agents; opalescent agents; ultra-violet stabilizers; anti-oxidants; viscosity modifiers, and the like can be added to the catalyst paste to impart desired properties to the composition.

A photoactive agent such as, for example, benzophenone, benzoin and their derivatives, or alpha-diketones and their derivatives can be added to the base or catalyst paste in order to make the composition light-curable. A preferred photopolymerization initiator is camphorquinone (CQ). Photopolymerization can be initiated by irradiating the composition with blue, visible light preferably having a wavelength in the range of about 400 to about 500 nm. A standard dental blue light-curing unit can be used to irradiate the composition. The camphorquinone (CQ) compounds have a light absorbency maximum of between about 400 to about 500 nm and generate free radicals for polymerization when irradiated with light having a wavelength in this range. Alternatively, the photoinitiator can be selected from the class of acylphosphine oxides such as monoacyl phosphine oxide derivatives, bisacyl phosphine oxide derivatives, and triacyl phosphine oxide derivatives. For, example, 2,4,6-trimethylbenzoyl-diphenyl-phosphine oxide (TPO) can be used as the photopolymerization initiator.

As discussed above, the base and catalyst pastes are preferably mixed together and dispensed using an auto-mix system such as a cartridge having dual chambers. The base and catalyst pastes are mixed together in a pre-determined volume ratio, preferably in a 1:1 volume ratio, to form a mixed composition. As the pastes are mixed together, the polymerization accelerator and initiator react with each other and initiate polymerization and hardening of the composition. The mixed pastes start polymerizing and hardening in less than about two (2) minutes from the beginning of the mixing. The viscosity of the mixed pastes gradually increases. In general, within about two (2) to about five (5) minutes from the beginning of the mixing, the composition is hardened.

The dental compositions can be used as crown and bridge materials, inlays, onlays, veneers, fillings, sealants, adhesives, cements, and other dental materials. More particularly, the composition can be used to make a temporary crown or bridge. The compositions have good shelf life, color stability, work time (WT)/set time (ST), physical properties, wear resistance and handling performance.

Particularly, the base and catalyst pastes each can be stored for extended periods of time and then mixed immediately prior to use. Because the base and catalyst pastes have good shelf-life, they can be handled and mixed effectively even though they have been stored for a substantial time period. The composition also has good color stability meaning that its color does not substantially change over time. As a result, the finished crown or other restoration can maintain its aesthetically-pleasing appearance and match the shade of surrounding teeth.

In addition, the composition had good working and setting times. By the term, “working time,” (WT) as used herein, it is meant the period of time from the initial mixing of the pastes to the point when the mixed composition begins to polymerize and form a gel-like material. The working time is measured as the time when the composition no longer peaks up from the surface of a working plate when probed by a sharp instrument. By the term, “setting time,” (ST) as used herein, it is meant the period of time from the period of time from the initial mixing of the pastes to the point when the mixed hardens sufficiently to form a material that can be snap-cut by a razor blade. In general, the materials of this invention have a working time in the range of about one (1) to about two (2) minutes. And, the setting time is generally within the range of about (1) to about (2) minutes longer than the working time. With this extended working time, the dental practitioner can shape and reshape the composition as needed to form a high quality and fitted restoration such as a temporary crown.

The present invention is further illustrated by the following Examples, but these Examples should not be construed as limiting the scope of the invention.

### EXAMPLES

Abbreviations:

Bis-GMA 2,2-bis[4(2-hydroxy-3-methacryloyloxypropyloxy)phenyl]propane
EBPADMA 2 mole ethoxylated bisphenol A dimethacrylate
UDMA urethane dimethacrylate
EBPADMA UR EBPADMA urethane resin
TEGDMA triethyleneglycol dimethacrylate
NANOCRYL D301 50 wt. % fumed silica filled TEGDMA
SR423 isobornyl methacrylate
SR444 pentaerythritol triacrylate
DEP diethyl phthalate
CHP cumene hydroperoxide
TMBP 1,1,3,3-tetramethyl butyl hydroperoxide
TAHP t-amyl hydroperoxide
TBHP t-butyl hydroperoxide
DMHP 2,5-dimethyl-2,5-di(hydroperoxy) hexane
DPMH diisopropylbenzene mono hydroperoxide
PMHP paramenthane hydroperoxide
MMIZ 2-mercapto-1-methylimidazole
STPB sodium tetraphenylborate
PTU 2-pyridylthiourea
ATU 1-acetyl-2-thiourea
PhTU phenyl thiourea
TU thiourea
BTU benzoylthiourea
AlTU 1-allyl-2-thiourea
AlHETU 1-allyl-3(-2-hydroxyethyl)-2-thiourea
CuAA copper actylacetonate
BHT butylated hydroxytoluene
BABG silanated barium boron aluminosilicate glass filler
EG9726 silanated fluoride barium boron aluminosilicate glass filler
TS720 fumed silica filler
WT Work Time
ST Set Time
GPP Gel Phase Period
CS Compressive Strength
TS Transverse Strength
FM Flexural Modulus
DTS Diametral Tensile Strength

### Examples 1-3

The sample compositions in Examples 1-3 were made with different two base pastes and three different catalyst pastes.

The two base pastes 1B and 2B were prepared by first dissolving 0.8 g PTU and 0.01 g BHT in a resin blend consisting of 27.19 g UDMA, 4.0 g DEP, and 8.0 g NANOCRYL D301 under mechanical stirring at 50° C. for one hour until a homogeneous solution was obtained. Subsequently, 57.0 g BABG and 3.0 g TS720 were added to form Paste 1B and 58.0 g BABG and 2.0 g TS720 were added independently to form Paste 2B. Each paste was prepared by mixing the components for 2 minutes using a SpeedMixer at room temperature.

The catalyst pastes 1C-3C were prepared individually by dissolving 1.6 g CHP and 0.03 g BHT in resin blends consisting of 32.37 EBPADMA, 6.0 NANOCRYL D301 (Paste IC); 25.26 g EBPADMA, 6.0 g Bis-GMA, 7.11 g NANOCRYL D301 (Paste 2C); and 26.37 g Bis-GMA, 12.0 NANOCRYL D301 (Paste 3C), respectively, under mechanical stirring for half hour at 50° C. Subsequently, 58.0 g BABG and 2.0 g TS720 were added to Paste 1C; 57.0 g BABG and 3.0 g TS720 were added to Paste 2C, and 59.0 g BABG and 1.0 g TS720 were added to Paste 3C, respectively. The pastes were prepared by mixing 2 minutes using a SpeedMixer at room temperature.

The resultant base and catalyst pastes were filled into 1:1 double barrel cartridges and the pastes were dispensed accordingly to form a mixed composition. The WT/ST of each sample was tested. The results are set forth in Table 1.

TABLE 1
Examples Demonstrating Effects of Different Resin Blends

| | | WT | ST |
|---|---|---|---|
| Example | Sample | (min) | (min) |
| 1 | 1C/1B | 2:35 | 3:15 |
| 2 | 2C/1B | 2:00 | 2:50 |
| 3 | 3C/2B | 1:20 | 1:50 |

### Examples 4-12

The sample compositions in Examples 4-12 were made with three base pastes and three catalyst pastes using different combinations.

The base pastes, 3B-5B were prepared by first dissolving 0.005 g BHT, an appropriate amount of PTU and MMIZ, as shown in Table 2, in resin blends consisting of 27.0 g UDMA (Paste 3B) 4.0 g TEGDMA (Paste 4B) and 8.0 g TEGDMA (Paste 5B), respectively under mechanical stirring at 50° C. for one hour until homogeneous resins were obtained. Subsequently, 63.0 g EG9726 and 2.0 g TS720 were added to each paste. The pastes were prepared by mixing the components for 2 minutes using a SpeedMixer at room temperature.

The catalyst pastes, 4C-6C were prepared by first dissolving 0.03 g BHT, an appropriate amount of CHP, as shown in Table 2, in a resin blend consisting of 20.97 g EBPADMA (Paste 4C), 4.0 Bis-GMA (Paste 5C), and 8.0 g TEGDMA/2.0 g SR444 (Paste 6C) under mechanical stirring for half hour at 50° C. Subsequently, 63.0 g EG9726 and 2.0 g TS720 were added to each paste. The pastes were prepared by mixing the components for 2 minutes using a SpeedMixer at room temperature.

The resultant base and catalyst pastes were filled into 1:1 double barrel cartridges and the pastes were dispensed accordingly to form a mixed composition. The WT/ST and mechanical properties of each sample was tested. The results are set forth in Table 2.

TABLE 2
Examples Demonstrating Effects of PTU, MMIZ and CHP Levels

| | | CHP | PTU | MMIZ | WT | ST | CS | TS | FM |
|---|---|---|---|---|---|---|---|---|---|
| Example | Sample | (g) | (g) | (g) | (min) | (min) | (MPa) | (MPa) | (MPa) |
| 4 | 4C/3B | 1.60 | 1.10 | 0.10 | 1:15 | 1:35 | 266 | 72 | 2782 |
| 5 | 4C/4B | 1.60 | 0.60 | 0.60 | 0:50 | 1:15 | 242 | 53 | 2512 |
| 6 | 4C/5B | 1.60 | 0.10 | 1.10 | 1:48 | 1:40 | 139 | 7 | 129 |
| 7 | 5C/3B | 1.20 | 1.10 | 0.10 | 1:20 | 1:50 | 287 | 70 | 2489 |
| 8 | 5C/4B | 1.20 | 0.60 | 0.60 | 1:00 | 1:25 | 248 | 69 | 2493 |
| 9 | 5C/5B | 1.20 | 0.10 | 1.10 | 1:10 | 2:10 | 113 | 12 | 239 |
| 10 | 6C/3B | 0.80 | 1.10 | 0.10 | 1:40 | 2:25 | 304 | 55 | 1997 |
| 11 | 6C/4B | 0.80 | 0.60 | 0.60 | 1:20 | 2:00 | 291 | 57 | 1916 |
| 12 | 6C/5B | 0.80 | 0.10 | 1.10 | 2:00 | 2:55 | 252 | 34 | 983 |

### Examples 13-31

The sample compositions in Examples 13-31 were made with different base pastes 6B-12B and the same catalyst paste 7C.

The base pastes 6B-12B were prepared by first dissolving 0.01 g BHT and an appropriate amount of different accelerator, as shown in Table 3, in a resin blend consisting of 26.79 g UDMA and 12.0 g NANOCRYL D301under mechanical stirring at 50° C. or one hour until a homogeneous solution was obtained. Subsequently, 58.0 g BABG and 2.0 g TS720 were added to the resin blends. Each paste was prepared by mixing the components for 2 minutes using a SpeedMixer at room temperature.

The catalyst paste 7C was prepared individually by dissolving 1.6 g CHP and 0.032 g BHT in resin blends consisting of 24.37 g Bis-GMA, 12.0 g NANOCRYL D301 and 2.0 g TEGDMA under mechanical stirring for half hour at 50° C. Subsequently, 59.0 g BABG and 1.0 g TS720 were added to each resin blend. The pastes were prepared by mixing the components for 2 minutes using a SpeedMixer at room temperature. The WT/ST of each sample was tested and the results are summarized in Table 3.

TABLE 3
Examples Demonstrating Effects of Different Reducers

| | | PTU | MMIZ | xTU | STPB | WT | ST | |
|---|---|---|---|---|---|---|---|---|
| Example | Sample | (g) | (g) | (g) | (g) | (min) | (min) | Note |
| 13 | 6B (PTU + MMIZ) | 0.60 | 0.60 | 0.00 | 0.00 | 0:45 | 1:00 | |
| 14 | 7B (no reducer) | 0.00 | 0.00 | 0.00 | 0.00 | N/A | N/A | no set |
| 15 | 8B (TU) | 0.00 | 0.00 | 0.80 | 0.00 | 6:00 | 7:10 | soft set |
| 16 | 9B (ATU) | 0.00 | 0.00 | 0.80 | 0.00 | 4:40 | 6:30 | |
| 17 | 10B (AITU) | 0.00 | 0.00 | 0.80 | 0.00 | 5:45 | 8:00 | |
| 18 | 11B (BTU) | 0.00 | 0.00 | 0.80 | 0.00 | 9:00 | 11:00 | |
| 19 | 12B (STPB) | 0.00 | 0.00 | 0.00 | 0.80 | N/A | N/A | no set > 10:00 |
| 20 | ½ 6B + ½ 7B | 0.30 | 0.30 | 0.00 | 0.00 | 1:15 | 1:50 | |
| 21 | ½ 6B + ½ 8B | 0.30 | 0.30 | 0.40 | 0.00 | 1:05 | 1:25 | |
| 22 | ½ 6B + ½ 9B | 0.30 | 0.30 | 0.40 | 0.00 | 1:10 | 1:30 | |
| 23 | ½ 6B + ½ 10B | 0.30 | 0.30 | 0.40 | 0.00 | N/A | 1:15 | gelled after mixing |
| 24 | ½ 6B + ½ 11B | 0.30 | 0.30 | 0.40 | 0.00 | 1:15 | 1:45 | |
| 25 | ½ 6B + ½ 128 | 0.30 | 0.30 | 0.00 | 0.40 | 0:45 | 1:15 | |
| 26 | ¼ 6B + ¾ 7B | 0.15 | 0.15 | 0.00 | 0.00 | 2:15 | 2:50 | |
| 27 | ¼ 6B + ¾ 8B | 0.15 | 0.15 | 0.60 | 0.00 | N/A | 1:15 | gelled after mixing |
| 28 | ¼ 6B + ¾ 9B | 0.15 | 0.15 | 0.60 | 0.00 | 2:00 | 2:40 | |
| 29 | ¼ 6B + ¾ 10B | 0.15 | 0.15 | 0.60 | 0.00 | 1:50 | 2:30 | |
| 30 | ¼ 6B + ¾ 11B | 0.15 | 0.15 | 0.60 | 0.00 | 2:40 | 3:35 | |
| 31 | ¼ 6B + ¾ 12B | 0.15 | 0.15 | 0.00 | 0.60 | 1:55 | 2:40 | |

### Examples 32-55

Examples 32-55 demonstrated the effects of using different combinations of reducing agents and initiators on the WT/ST of the compositions.

The base pastes 20B-25B were prepared by first dissolving 0.005 g BHT, 0.01 g of CuAA and an appropriate amount of different accelerator, as shown in Table 4, in a resin blend consisting of the following resin combinations: 14.46 g EBPADMA UR, 20.02 g UDMA and 8.7 g SR423 (Sample 20B); 6.89 g EBPADMA, and 12.29 g EBPADMA UR, 22.13 g UDA and 7.87 g SR423 (Samples 21B-22B); 21.12 g EBPADMA UR, 21.12 g UDMA and 6.75 g SR423 (Samples 23B-25B). A clear solution was obtained after mechanically stirred for one hour at 50° C. for each resin blend. Subsequently, 47.0 g BABG and 3.0 g TS720 were added to the resin blend, each paste was prepared by mixing 2 minutes using SpeedMixer at room temperature.

The catalyst paste 8C-11C were prepared by first dissolving 0.03 g BHT and 2.0 g different initiators in a resin blend consisting of 23.97 g EBPADMA, 14.4 g Bis-GMA, 6.72 g TEGDMA and 2.88 g SR444 under mechanical stirring for half hour at 50° C. Subsequently, 47.0 g BABG and 3.0 g TS720 were added to the resin blend, each paste was prepared by mixing 2 minutes using SpeedMixer at room temperature. The WT/ST of each sample was tested and the results are summarized in Table 4.

TABLE 4
Examples Demonstrating the Effect of Different Redox Agents

| | | xTU | xHP | WT | ST |
|---|---|---|---|---|---|
| Example | Sample | (g) | (g) | (min) | (min) |
| 32 | 20B/8C | 0.8 PTU | 2.0 CHP | 0:30 | 1:00 |
| 33 | 21B/8C | 0.8 AITU | | 1:15 | 1:50 |
| 34 | 22B/8C | 0.8 AIHETU | | 1:50 | 2:45 |
| 35 | 23B/8C | 1.0 BTU | | 3″18 | 5:23 |
| 36 | 24B/8C | 1.0 ATU | | 0:40 | 0:55 |
| 37 | 25B/8C | 1.0 PhTU | | 0:50 | 1:00 |
| 38 | 20B/9C | 0.8 PTU | 2.0 TMBP | 0:30 | 1:00 |
| 39 | 21B/9C | 0.8 AITU | | 1:10 | 1:40 |
| 40 | 22B/9C | 0.8 AIHETU | | 1:25 | 2:00 |
| 41 | 23B/9C | 1.0 BTU | | 2:40 | 4:30 |
| 42 | 24B/9C | 1.0 ATU | | 0:50 | 1:30 |
| 43 | 25B/9C | 1.0 PhTU | | 0:50 | 1:33 |
| 44 | 20B/10C | 0.8 PTU | 2.0 TAHP | 1:20 | 2:45 |
| 45 | 21B/10C | 0.8 AITU | | 2:00 | 4:30 |
| 46 | 22B/10C | 0.8 AIHETU | | 2:00 | 4:00 |
| 47 | 23B/10C | 1.0 BTU | | 5:30 | 10:00 |
| 48 | 24B/10C | 1.0 ATU | | 1:18 | 3:00 |
| 49 | 25B/10C | 1.0 PhTU | | 1:30 | 3:30 |
| 50 | 20B/11C | 0.8 PTU | 2.0 TBHP | 2:00 | 5:00 |
| 51 | 21B/11C | 0.8 AITU | | 3:25 | 8:00 |
| 52 | 22B/11C | 0.8 AIHETU | | 3:05 | 7:00 |
| 53 | 23B/11C | 1.0 BTU | | 7:45 | 15:00 |
| 54 | 24B/11C | 1.0 ATU | | 2:05 | 5:00 |
| 55 | 25B/11C | 1.0 PhTU | | 2:45 | 8:00 |

### Examples 56-58

Examples 56-58 demonstrated effects of different CuAA levels on the WT/ST of the compositions.

Catalyst paste 8C was used for Examples 56-58. The base pastes 26B-28B were prepared by first dissolving 0.005 g BHT, 1.0 g ATU and an appropriate amount of CuAA, as shown in Table 5, in a resin blend under mechanical stirring for one hour at 50° C., resin composition was identical to that described in Example 36. Subsequently, 47.0 g BABG and 3.0 g TS720 were added to the resin blends. Each paste was prepared by mixing 2 minutes using SpeedMixer at room temperature. The WT/ST of each sample was tested and compared with the sample in Example 36. The results are set forth in Table 5.

TABLE 5
Examples Demonstrating Effects of CuAA Level

| | | CuAA | WT | ST |
|---|---|---|---|---|
| Example | Sample | (g) | (min) | (min) |
| 36 | 24B/8C | 0.0100 | 0:40 | 0:55 |
| 56 | 26B/8C | 0.0050 | 0:55 | 1:05 |
| 57 | 27B/8C | 0.0025 | 1:35 | 2:00 |
| 58 | 28B/8C | 0.0000 | 5:00 | 7:30 |

### Examples 59-62

Examples 59-62 demonstrated the effects of using a blend of CHP and TAHP at weight ratios on the WT/ST of the compositions.

Base paste 26B was used for Examples 59-62. The catalyst pastes 12C-14C were prepared by first dissolving 0.03 g BHT and an appropriate amount of CHP and TAHP in a resin blend under mechanical stirring for half hour at 50° C. The resin composition was identical to that described in Example 32. Subsequently, 47.0 g BABG and 3.0 g TS720 were added to each resin blend, and the pastes were prepared by mixing for 2 minutes using a SpeedMixer at room temperature. The WT/ST of each sample was tested and compared with the sample in Example 56. The results are set forth in Table 6.

TABLE 6
Examples Containing Different TAHP and CHP Combinations

| | | xHP | WT | ST |
|---|---|---|---|---|
| Example | Sample | (g) | (min) | (min) |
| 59 | 26B/10C | 2.0 TAHP | 2:00 | 4:00 |
| 60 | 268/12C | 1.5 TAHP | 1:40 | 3:40 |
| | | 0.5 CHP | | |
| 61 | 26B/13C | 1.33 TAHP | 1:40 | 3:00 |
| | | 0.67 CHP | | |
| 62 | 26B/14C | 1.0 TAHP | 1:30 | 2:25 |
| | | 1.0 CHP | | |
| 56 | 26B/8C | 2.0 CHP | 0:55 | 1:05 |

### Examples 63-67

Examples 63-67 demonstrated the effects of using different redox combinations on the WT/ST of the compositions.

Base paste 29B was prepared by first dissolving 0.75 g ATU, 0.0075 g CuAA and 0.005 g BHT in a resin blend which was identical to that described in Example 36; and base paste 30B was prepared by first dissolving 1.0 g PTU, 0.005 g CUAA and 0.01 g BHT in resin blend which was identical to that described in Example 32. A clear solution was obtained after mechanically stirred for one hour at 50° C. for each resin blend. Subsequently, 47.0 g BABG and 3.0 g TS720 were added to each resin blend, and the pastes were prepared by mixing 2 minutes using a SpeedMixer at room temperature.

The catalyst pastes 15C and 16C were prepared by first dissolving 0.03 g BHT and an appropriate amount of TAHP and TMBH in a resin blend under mechanical stirring for half hour at 50° C. with the resin composition being identical to that composition described in Examples 32. Subsequently, 47.0 g BABG and 3.0 g TS720 were added to each resin blend, and the pastes were prepared by mixing 2 minutes using SpeedMixer at room temperature. The WT/ST for each sample was tested and the results are set forth in Table 7.

TABLE 7
Examples Containing Different Redox Combinations

| | | xTU | CuAA | xHP | WT | ST |
|---|---|---|---|---|---|---|
| Example | Sample | (g) | (g) | (g) | (min) | (min) |
| 63 | 29B/12C | 0.75 ATU | 0.0075 | 1.5 TAHP | 1:30 | 2:50 |
| | | | | 0.5 CHP | | |
| 64 | 25B/12C | 1.0 PHTU | 0.0100 | 1.5 TAHP | 1:15 | 2:30 |
| | | | | 0.5 CHP | | |
| 65 | 30B/15C | 1.0 PTU | 0.0050 | 1.0 TMBH | 1:12 | 2:00 |
| 66 | 20B/16C | 0.8 PTU | 0.0100 | 0.5 TMBH | 1:10 | 2:20 |
| | | | | 0.5 TAHP | | |
| 67 | 20B/10C | 0.8 PTU | 0.0100 | 2.0 TAHP | 1:20 | 2:45 |

Table 8 describes the mechanical properties of compositions from selected Examples as described above.

TABLE 8
Mechanical Properties of Selected Examples

| | CS | TS | FM | DTS |
|---|---|---|---|---|
| Example | (MPa) | (MPa) | (MPa) | (MPa) |
| 1 | 292 | 62 | 1945 | 37 |
| 2 | 283 | 69 | 2402 | 30 |
| 3 | 274 | 63 | 1933 | 32 |
| 34 | 285 | 47 | 990 | 43 |
| 48 | 270 | 87 | 3208 | 50 |
| 56 | 282 | 98 | 3188 | 54 |
| 64 | 249 | 66 | 1608 | 42 |
| 65 | 273 | 84 | 2555 | 52 |
| 66 | 243 | 63 | 1512 | 41 |

What is claimed is:

1. A polymerizable dental composition, comprising:
a polymerizable compound, wherein said compound does not contain any acidic groups;
a polymerization system for initiating polymerization of the composition, the system comprising a thiourea compound and hydroperoxide compound.

2. The dental composition of claim 1, wherein the polymerizable compound is selected from the group consisting of 2,2-bis[4-(2-hydroxy-3-methacryloyloxypropoxy)phenyl]propane; ethoxylated bisphenol A-dimethacrylate); urethane di(meth)acrylate; diurethane dimethacrylate; polyurethane dimethacrylate; tri(ethylene glycol) dimethacrylate; and mixtures thereof.

3. The dental composition of claim 1, wherein the thiourea compound is selected from the group consisting of thiourea; 2-pyridyl thiourea; phenolthiourea; 1-acetyl-2-thiourea; 1-allyl-2-thiourea; 1-allyl-3-(2hydroxyethyl)-2-thiourea; benzoyl thiourea; and mixtures thereof.

4. The dental composition of claim 1, wherein the hydroperoxide compound is selected from the group consisting of cumene hydroperoxide; t-amyl-hydroperoxide; 1,1,3,3-tetramethyl butyl hydroperoxide; t-butyl-hydroperoxide; 2,5-dimethyl-2,5-di(hydroperoxy)hexane; diisopropylbenzene mono hydroperoxide; paramenthane hydroperoxide; and mixtures thereof.

5. The dental composition of claim 1, wherein the composition comprises a blend of cumene hydroperoxide and t-amyl-hydroperoxide compounds.

6. The dental composition of claim 1, wherein the polymerization system comprises a mixture of 2-pyridyl thiourea; 2-mercapto-1-methylimidazole; and hydroperoxide compound.

7. The dental composition of claim 1, wherein the polymerization system comprises a mixture of 2-pyridyl thiourea; sodium tetraphenyl borate; and hydroperoxide compound.

8. The dental composition of claim 1, wherein the polymerization system comprises a mixture of thiourea compounds; metal-containing species; and hydroperoxide compound.

9. The dental composition of claim 8, wherein the metal-containing species is copper acetylacetonate.

10. The dental composition of claim 1, further comprising filler material.

11. The dental composition of claim 10, wherein the filler material is selected from the group consisting of silica, alumina, titanium dioxide, iron oxides, silicon nitrides, glasses, kaolin, quartz, talc, and mixtures thereof.

12. The dental composition of claim 10, wherein the filler material comprises aluminosilicate glass.

13. The dental composition of claim 1, further comprising a photoactive agent for making the composition light-curable.

14. The dental composition of claim 1, wherein the composition is in the form of two parts, the first part comprising the thiourea compound and the second part comprising the hydroperoxide compound.

15. The dental composition of claim 14, wherein the first part comprising the thiourea compound is a base paste and the second part comprising the hydroperoxide compound is a catalyst paste.
